(19) United States
(12) Patent Application Publication
(10) Pub. No.: US 2026/0191580 A1
(43) Pub. Date: Jul. 9, 2026

(54) LACERATION SYSTEM AND DEVICE, AND METHODS FOR LACERATION

(71) Applicant: Boston Scientific Medical Device Limited, Galway (IE)

(72) Inventors: Eduardo Moriyama, Richmond (CA); Jackie Leung, Richmond Hill (CA); Gareth Davies, Toronto (CA); Kaylie Lau, Toronto (CA)

(73) Assignee: Boston Scientific Medical Device Limited, Galway (IE)

(21) Appl. No.: 19/559,288

(22) Filed: Mar. 6, 2026

Related U.S. Application Data
(63) Continuation of application No. 17/996,238, filed on Apr. 14, 2021, now abandoned.
(60) Provisional application No. 63/013,604, filed on Apr. 22, 2020.

(51) Int. Cl. A61B 18/14; A61B 18/00

(52) U.S. Cl. CPC A61B 18/1445; A61B 2018/00059; A61B 2018/00601

(57) ABSTRACT

A laceration device for use in medical procedures includes a shaft having opposed proximal and distal portions. A clamp extends from the distal portion. The clamp has ftrst and second clamp arms. The clamp is movable between an open and closed positions. In the open position, the first clamp arm is spaced apart from the second clamp arm. In the closed position, the first clamp arm is moved towards the second clamp arm. A clamp actuator is connected to the clamp via the shaft and is manipulatable to move the clamp between the open and closed positions. A radiofrequency electrode (RF) is associated with the fast clamp arm. The RF electrode has a first perforation surface that is positioned to face the second clamp arm when the clamp is in the closed position. An electrical connector extends proximally from the RF electrode for connection to a power source.

### CROSS-REFERENCE TO RELATED APPLICATIONS

This application is a continuation of U.S. Application No., 17/996,238, filed October 14, 2022, which is a §371 of PCT/IB2021/053085, filed April 14, 2021, which claims benefit of US Provisional Application No. 63/013,604, filed April 22, 2020, all of which are incorporated by reference.

### BACKGROUND/SUMMARY

### FIELD

This document relates to medical procedures that involve laceration of an anatomical structure. More specifically, this document relates to devices for laceration, and related systems and methods.

### SUMMARY

The following summary is intended to introduce the reader to various aspects of the detailed description, but not to define or delimit any invention.

Laceration devices for use in medical procedures are disclosed. According to some aspects, a laceration device includes a shaft having a proximal portion and an opposed distal portion. A clamp extends from the distal portion of the shaft. The clamp has at least a first clamp arm and a second clamp arm. The clamp is movable between an open position and a closed position. In the open position, the first clamp arm is spaced apart from the second clamp arm. In the closed position, the first clamp arm is moved towards the second clamp arm relative to the open position. A clamp actuator is connected to the clamp via the shaft and is manipulatable to move the clamp between the open position and the closed position. At least a first radiofrequency electrode is associated with the first clamp arm. The first radiofrequency electrode has a first perforation surface that is positioned to face the second clamp arm when the clamp is in the closed position. An electrical connector extends proximally from the first radiofrequency electrode for connection to a power source.

In some examples, the first perforation surface is spaced from the clamp arm, and the first radiofrequency electrode further includes a first laceration surface that is proximal of and transverse to the first perforation surface, and extends towards the first clamp arm from the first perforation surface

In some examples, the laceration device further includes a second radiofrequency electrode associated with the second clamp arm. The second radiofrequency electrode can have a second perforation surface that is positioned to face the first perforation surface when the clamp is in the closed position.

In some examples, when the clamp is in the closed position, the first perforation surface and second perforation surface are in contact.

In some examples, the first clamp arm and second clamp arm each have, respectively, an inner end portion secured to the shaft and an outer end portion opposite the inner end portion. The first laceration electrode can be associated with the outer end portion of the first clamp arm. Alternatively, the first laceration electrode can extend along the inner end portion and the outer end portion of the first clamp arm.

In some examples, at least a portion of the clamp is radiopaque.

Methods for creating a laceration are also disclosed. According to some aspects, a method for creating a laceration includes a) advancing a clamp of a laceration device towards a target anatomical structure; b) after step a), clamping the clamp onto the target anatomical structure to secure the clamp to the target anatomical structure and position a first radiofrequency electrode of the clamp against a first surface of the target anatomical structure; and c) activating the first radiofrequency electrode to lacerate the target anatomical structure.

In some examples, clamping the clamp onto the target anatomical structure further positions a second laceration electrode of the clamp against a second surface of the target anatomical structure. The second surface can be opposite the first surface.

In some examples, step c) further includes activating the second laceration electrode.

In some examples, step c) further includes, with the first radiofrequency electrode activated, applying force to move the first radiofrequency electrode along the target anatomical structure.

In some examples, step b) includes manipulating a clamp actuator to clamp the clamp onto the target anatomical structure.

Laceration systems for use in medical procedures are also disclosed. According to some aspects, a laceration system for use in medical procedures includes a radiofrequency generator and a laceration device. The laceration device includes a shaft having a proximal portion and an opposed distal portion. A clamp extends from the distal portion of the shaft. The clamp has at least a first clamp arm and a second clamp arm. The clamp is movable between an open position and a closed position. In the open position, the first clamp arm is spaced apart from the second clamp arm. In the closed position, the first clamp arm is moved towards the second clamp arm relative to the open position. A clamp actuator is connected to the clamp via the shaft and is manipulatable to move the clamp between the open position and the closed position. At least a first radiofrequency electrode is associated with the first clamp arm. The first radiofrequency electrode has a first perforation surface that is positioned to face the second clamp arm when the clamp is in the closed position. An electrical connector connects the first radiofrequency electrode to the radiofrequency generator.

In some examples, the first perforation surface is spaced from the first clamp arm, and the first radiofrequency electrode further includes a first laceration surface that is proximal of and transverse to the first perforation surface and extends towards the first clamp arm from the first perforation surface

In some examples, the laceration system further includes a second radiofrequency electrode associated with the second clamp arm and electrically connected to the radiofrequency generator. The second radiofrequency electrode can have a second perforation surface that is positioned to face the first perforation surface when the clamp is in the closed position.

In some examples, when the clamp is in the closed position, the first perforation surface and second perforation surface are in contact.

In some examples, the first clamp arm and second clamp arm each have, respectively, an inner end portion secured to the shaft and an outer end portion opposite the inner end portion. The first laceration electrode can be associated with the outer end portion of the first clamp arm. Alternatively, the first laceration electrode can extend along the inner end portion and the outer end portion of the first clamp arm.

In some examples, at least a portion of the clamp is radiopaque.

### DESCRIPTION

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of an example laceration system;

FIG. 2A is a side view of the laceration device of the laceration system of FIG. 1, showing a clamp thereof in an open position;

FIG. 2B is an enlarged cross-sectional view of the encircled region of FIG. 2A;

FIG. 3 is a side view of the laceration device of the laceration system of FIG. 1, showing a clamp thereof in a closed position;

FIG. 4 is a partial side view of the clamp arms of the laceration device of FIGS. 2A to 3, showing the clamp in the closed position;

FIG. 5 is a cross-section taken through the clamp arms of FIG. 4;

FIG. 6 is schematic view showing a first step of a method for creating a laceration;

FIG. 7 is a schematic view showing a subsequent step of the method of FIG. 6;

FIG. 8 is a schematic view showing a subsequent step of the method of FIG. 7; and

FIG. 9 is a schematic view showing a subsequent step of the method of FIG. 8.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are devices, and related systems and methods, that can be used in medical procedures in which a target anatomical structure is lacerated. Such medical procedures can include transvenous structural heart procedures, which can involve the laceration of soft tissue. Such soft tissue can include, for example, a valve leaflet or an atrial septum. The devices disclosed herein can clamp on to the target anatomical structure, perforate the target anatomical structure, and lacerate the target anatomical structure.

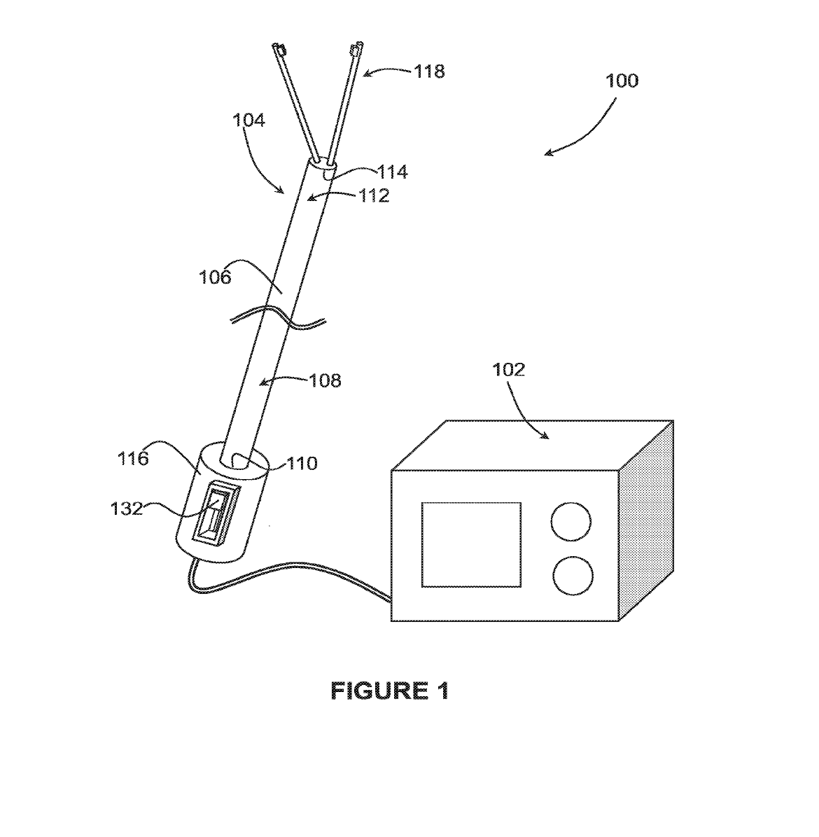

Referring now to FIG. 1, an example laceration system 100 is shown. The system 100 includes a power source in the form of a radiofrequency (RF) generator 102, as well as a laceration device 104. The laceration device 104 is electrically connectable to the RF generator 102, to supply RF energy to electrodes (described below) of the laceration device 104. The system 100 as shown in FIG. 1 is designed for operation in monopolar mode; however, in alternative examples, the system can operate in bipolar mode. In examples wherein the system orates in monopolar mode, the system can also include one or more grounding pads (not shown) connected to the RF generator.

Referring still to FIG. 1, in the example shown, the laceration device 104 includes an elongate shaft 106, which has a proximal portion 108 defining a proximal end 110 and a distal portion 112 defining a distal end 114. The shaft can optionally be fixed or steerable. A handle 116 is connected to the proximal end 110 of the shaft 106. The handle 116 can optionally include various controls, e.g. for controlling delivery of RF energy from the generator or for manipulating a clamp of the device (described below). The shaft 106 can be of various constructions. For example, the shaft 106 can be in the form of a plastic tube that houses various other components (e.g. electrical wires and mechanical components of the device).

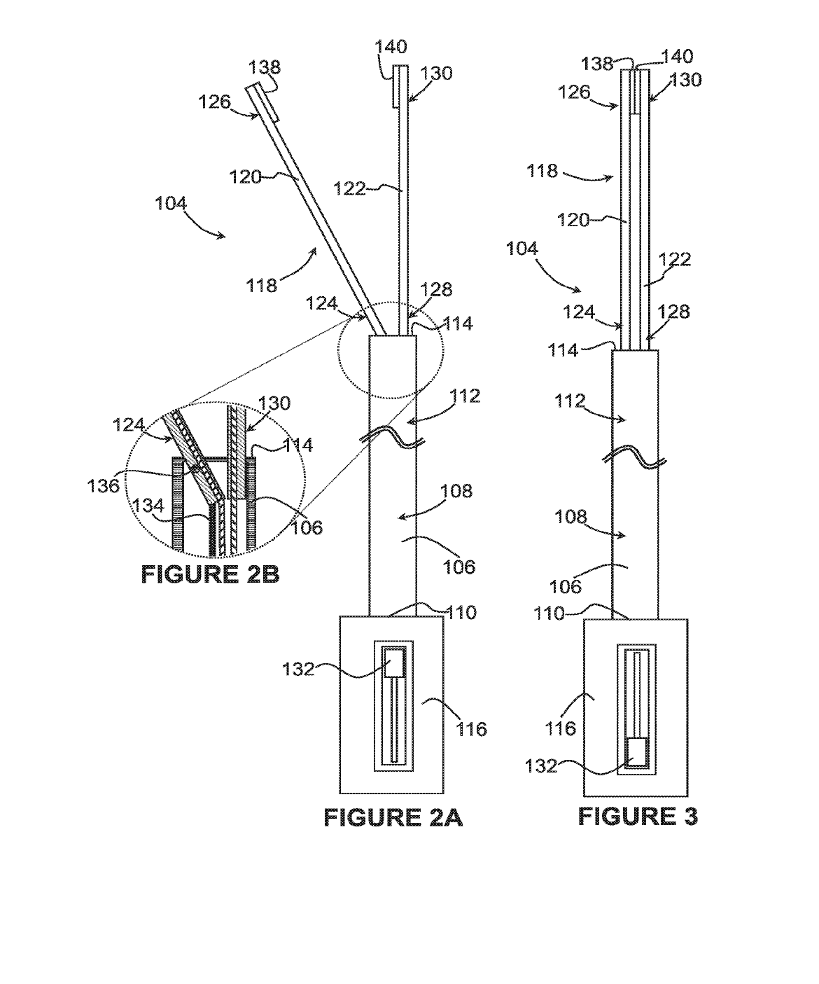

Referring now to FIGS. 2A, 2B, and FIG. 3, a clamp 118 extends from the distal portion 112 of the shaft 106. The clamp 118 has a first clamp arm 120 and a second clamp arm 122. The first clamp arm 120 has an inner end portion 124 secured to the shaft 106 and an outer end portion 126 that is opposite the inner end portion 124. Similarly, the second clamp arm 122 has an inner end portion 128 secured to the shaft 106 and an outer end portion 130 that is opposite the inner end portion 128. The clamp 118 is movable between an open position, shown in FIGS. 2A and 2B, and a closed position, shown in FIG. 3. In the open position, the first clamp arm 120 is spaced apart from the second clamp arm 122. In the closed position, relative to the open position, the first clamp arm 120 is moved toward the second clamp arm 122. More specifically, in the open position, the outer end portions 126, 130 of the clamp arms 120, 122 are spaced apart, and in the closed position, the outer end portions 126, 130 of the clamp arms 120, 122 are moved towards each other.

The open position may include angles other than that shown in FIGS. 2A and 2B. For example, when the clamp is in the open position, the claim arms may be spaced apart by 180 degrees.

Referring still to FIGS. 2A, 2B, and FIG. 3, in the example shown, the laceration device 104 includes a clamp actuator that is on the handle 116 and is connected to the clamp 118 via the shaft 106. The clamp actuator can be manipulated to move the clamp 118 between the open position and the closed position. More specifically, referring to FIGS. 2A and 2B, in the example shown, the clamp actuator is in the form of a slide 132 that is connected to the inner end portion 124 of the first clamp arm 120 via a pull wire 134 that extends through the shaft 106 between the first clamp arm 120 and the slide 132. The first clamp arm is pivotable about a pivot pin 136, and the second clamp arm 122 is stationary. By sliding the slide 132 proximally, the inner end portion 124 of the first clamp arm 120 is pulled proximally, to force the first clamp arm 120 to pivot about the pivot pin 136 to the closed position. By sliding the slide 132 distally, the inner end portion 124 of the first clamp arm 120 is pushed distally, to force the first clamp arm 120 to pivot about the pivot pin 136 back to the open position. Optionally, a spring or other biasing member (not shown) can be provided to bias the clamp 118 in the closed position or the open position.

In alternative examples, both first clamp arm and the second clamp arm can be movable, or the second clamp arm can be movable while the first clamp arm can be stationary.

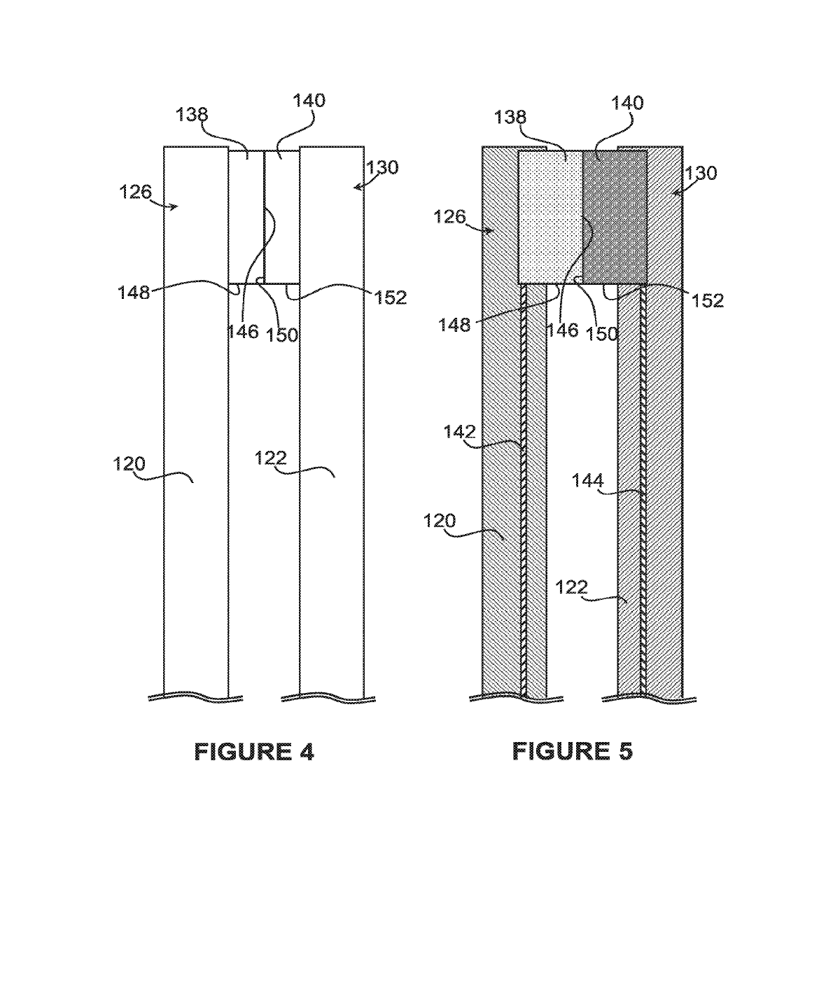

Referring now to FIGS. 4 and 5, in the example shown, each clamp arm 120, 122 is provided with a radiofrequency electrode—that is, a first radiofrequency electrode 138 is associated with the outer end portion 126 of the first clamp arm 120 and a second radiofrequency electrode 140 is associated with the outer end portion 130 of the second clamp arm 122. As used herein, the term ‘associated with’ means that the first referenced part (i.e. in this case the radiofrequency electrodes 138, 140, respectively) and second referenced part (i.e. in this case the clamp arms 120, 122, respectively) are configured so that the first referenced part moves with the second referenced part. For example, the first referenced part can be mounted to, extend from, adhered to, embedded in, part of, formed by, and/or integral with second referenced part. A first electrical connector 142 (e.g. a first insulated wire) extends proximally from the first radiofrequency electrode 138, through the first clamp arm 120 and the shaft 106 (not shown in FIGS. 4 and 5), for connection to the RF generator 120 (not shown in FIGS. 4 and 5) via the handle 116 (not shown in FIGS. 4 and 5), and a second electrical connector 144 (e.g. a second insulated wire) extends proximally from the second radiofrequency electrode 140, through the second clamp arm 122 and the shaft 106, for connection to the RF generator 102, via the handle 116. Supply of RF energy from the RF generator 102 to the RF electrodes 138, 140 causes the RF electrodes 138, 140 to cut (i.e. perforate or lacerate, as described below) tissue.

In alternative examples, the first and second radiofrequency electrodes may share wiring and may be activated concurrently.

Referring still to FIGS. 4 and 5, in the example shown, each radiofrequency electrode 138, 140 has a pair of cutting surfaces. Namely, the first radiofrequency electrode 138 has a perforation surface 146 (also referred to herein as a ‘first perforation surface), and a laceration surface 148 (also referred to herein as a ‘first laceration surface’). The first perforation surface 146 is positioned to face the second clamp arm 122 when the clamp 118 is in the closed position, and is spaced from the first clamp arm 120. The first laceration surface 148 is proximal of and transverse to the first perforation surface 146, and extends back towards the first clamp arm 120 from the first perforation surface 146. The second radiofrequency electrode 140 also has a perforation surface 150 (also referred to herein as a ‘second perforation surface’) and a laceration surface 152 (also referred to herein as a ‘second laceration surface’), which are configured similarly to the first perforation surface 146 and the first laceration surface 148. In the example shown, when the clamp 118 is in the closed position, the first perforation surface 146 and second perforation surface 150 face each other and are in contact.

In the example shown, the radiofrequency electrodes 138, 140 are relatively short in length compared to the clamp arms 120, 122, and are associated with the outer end portions 126, 130 of the clamp arms 120, 122. In alternative examples (not shown), the radiofrequency electrodes can be relatively long, so that they extend along the both the inner end portion and outer end portion of the first and second clamp arms, respectively.

Optionally, at least a portion of the clamp can be radiopaque. For example, the clamp arms can include a radiopaque marker (not shown) proximate the radiofrequency electrodes, or the clamp arms can be constructed of a radiopaque material, for viewing the clamp arms under fluoroscopy.

In the example shown, the clamp arms are generally straight. In alternative examples (not shown), the clamp arms can be curved or bent.

In alternative examples of a laceration device (not shown), the clamp can include only a single radiofrequency electrode. That is, the first clamp arm can include a radiofrequency electrode, and a second clamp arm can be electrically neutral.

Optionally, the clamp and/or catheter can be configured to inject a fluid (e.g. dextrose or another non-ionic fluid to electrically isolate the electrode from the surroundings). For example, the clamp and/or catheter can include a lumen that extends to the handle to allow for the injection of fluid.

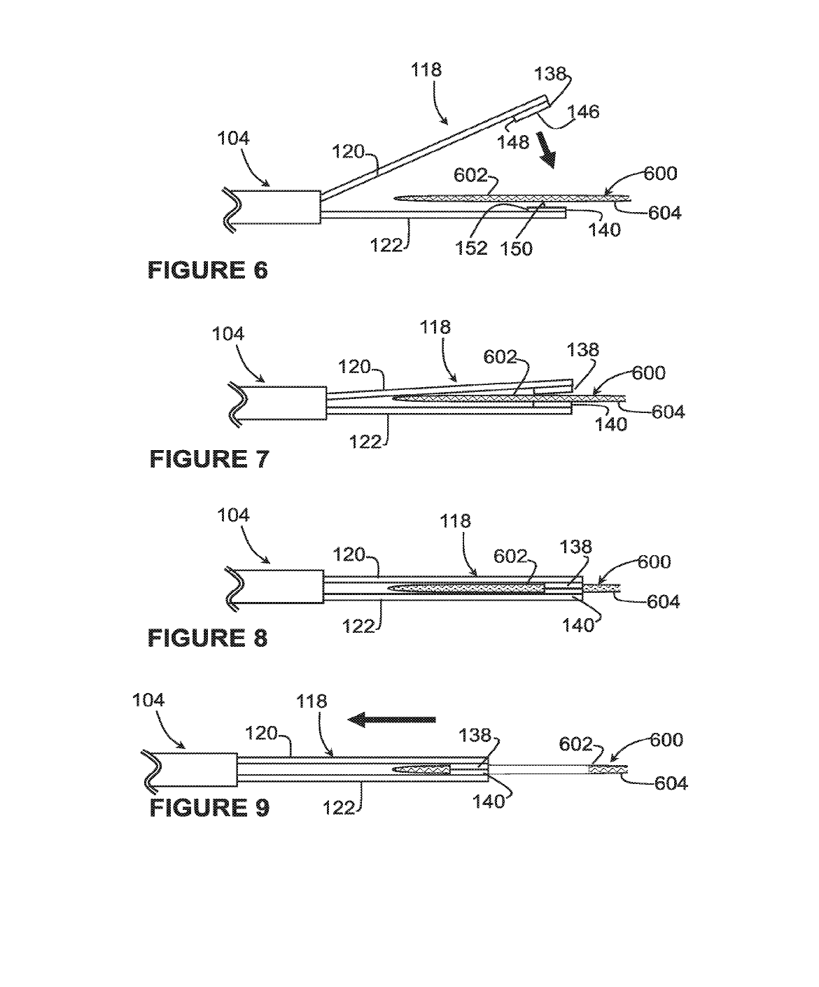

Referring now to FIGS. 6 to 9, in use, the laceration device 104 can be used to lacerate a target anatomical structure 600. For simplicity, the perforation surfaces 146, 148 and laceration surfaces 150, 152 are labelled only in FIG. 6, and not in FIGS. 7 to 9. Referring first to FIG. 6, the laceration device 104 can be advanced (e.g. advanced intravenously via a sheath) towards the target anatomical structure 600, and with the clamp 118 in the open position, the clamp 118 can be positioned so that the target anatomical structure 600 is between the clamp arms 120, 122. Referring to FIG. 7, the clamp 118 can then be moved towards the closed position by manipulating the slide 132 (not shown in FIGS. 6 to 9), so that clamp 118 is clamped onto the target anatomical structure 600. Clamping onto the target anatomical structure positions the radiofrequency electrodes 138, 140, and more specifically the respective perforation surfaces 146, 150, against opposing first 602 and second 604 surfaces of the target anatomical structure 600. Referring to FIG. 8, the radiofrequency electrodes 138, 140 can then be activated (i.e. by supplying RF energy from the RF generator 102, not shown in FIGS. 6 to 9), which causes the respective perforation surfaces 146, 150 of the radiofrequency electrodes 138, 140 to cut and perforate the target anatomical structure 600. When the radiofrequency electrodes 138, 140 have perforated the target anatomical structure 600—i.e. when the perforation surfaces 146, 150 are in contact with each other—force can be applied to move the laceration device 104 along the target anatomical structure 600, with the radiofrequency electrodes 138, 140 still activated. For example, the laceration device 104 can be pulled using the handle 116 (not shown in FIGS. 6 to 9). Referring to FIG. 9, the application of force causes the laceration surfaces 148, 152 to cut and lacerate the target anatomical structure 600.

After laceration is complete, the radiofrequency electrodes 138, 140 can be de-activated (e.g. by turning off the RF generator 102). If the target anatomical structure 600 is lacerated so that the target anatomical structure 600 is splayed into two sections (not shown), then the clamp 118 may remain in the closed position and the laceration device 104 can be withdrawn from the patient's body. Alternatively, if the target anatomical structure 600 is not splayed into two sections, the clamp 118 can be moved to the open position to release it from the target anatomical structure 600, then moved away from and clear of the target anatomical structure 600, then moved back to the closed position, so that the laceration device 104 can then be withdrawn from the patient's body. Alternatively, if the clamp can be moved to an open position in which the clamp arms are spaced apart by 180 degrees or close to 180 degrees, the laceration device can be withdrawn while the clamp is in the open position.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

What is claimed is:

1. A method for creating a laceration in a target anatomical structure, comprising:
advancing a laceration device towards the target anatomical structure, the laceration device comprising a shaft having a proximal portion and an opposed distal portion, a clamp extending from the distal portion of the shaft, the clamp having at least a first clamp arm and a second clamp arm, a clamp actuator connected to the clamp via the shaft, a first radiofrequency electrode associated with the first clamp arm, and a second radiofrequency electrode associated with the second clamp arm;
positioning the clamp such that the target anatomical structure is between the first clamp arm and the second clamp arm while the clamp is in an open position;
manipulating the clamp actuator to move the clamp from the open position to a closed position, wherein moving the clamp to the closed position causes the first clamp arm to move towards the second clamp arm to clamp onto the target anatomical structure;
activating at least the first radiofrequency electrode to perforate the target anatomical structure; and
applying force to move the first radiofrequency electrode along the target anatomical structure while the first radiofrequency electrode is activated to lacerate the target anatomical structure.

2. The method of claim 1, wherein moving the clamp to the closed position positions a first perforation surface of the first radiofrequency electrode against a first surface of the target anatomical structure.

3. The method of claim 2, wherein moving the clamp to the closed position further positions a second perforation surface of the second radiofrequency electrode against a second surface of the target anatomical structure, wherein the second surface is opposite the first surface.

4. The method of claim 3, wherein activating at least the first radiofrequency electrode comprises activating both the first radiofrequency electrode and the second radiofrequency electrode.

5. The method of claim 4, wherein activating both the first radiofrequency electrode and the second radiofrequency electrode causes the first perforation surface and the second perforation surface to perforate the target anatomical structure until the first perforation surface and the second perforation surface are in contact with each other.

6. The method of claim 1, wherein applying force to move the first radiofrequency electrode along the target anatomical structure comprises moving a first laceration surface of the first radiofrequency electrode along the target anatomical structure, wherein the first laceration surface is transverse to a first perforation surface of the first radiofrequency electrode.

7. The method of claim 6, wherein applying force to move the first radiofrequency electrode along the target anatomical structure further comprises moving a second laceration surface of the second radiofrequency electrode along the target anatomical structure, wherein the second laceration surface is transverse to a second perforation surface of the second radiofrequency electrode.

8. The method of claim 1, wherein manipulating the clamp actuator comprises manipulating a slide connected to the first clamp arm via a pull wire extending through the shaft.

9. A method for lacerating tissue in a medical procedure, comprising:
providing a laceration system comprising a radiofrequency generator and a laceration device, the laceration device comprising a shaft, a clamp extending from a distal portion of the shaft and having a first clamp arm and a second clamp arm, a first radiofrequency electrode associated with the first clamp arm and including a first perforation surface and a first laceration surface, and a second radiofrequency electrode associated with the second clamp arm and including a second perforation surface and a second laceration surface;
advancing the laceration device towards a target anatomical structure;
clamping the clamp onto the target anatomical structure to position the first perforation surface against a first surface of the target anatomical structure and to position the second perforation surface against a second surface of the target anatomical structure opposite the first surface;
supplying radiofrequency energy from the radiofrequency generator to the first radiofrequency electrode and the second radiofrequency electrode to cause the first perforation surface and the second perforation surface to perforate through the target anatomical structure; and
while continuing to supply radiofrequency energy, applying force to move the laceration device such that the first laceration surface and the second laceration surface lacerate the target anatomical structure.

10. The method of claim 9, wherein clamping the clamp onto the target anatomical structure comprises moving the clamp from an open position to a closed position.

11. The method of claim 10, wherein supplying radiofrequency energy causes the first perforation surface and the second perforation surface to perforate through the target anatomical structure until the first perforation surface and the second perforation surface are in contact with each other.

12. The method of claim 9, wherein the first laceration surface is proximal of and transverse to the first perforation surface and extends towards the first clamp arm from the first perforation surface.

13. The method of claim 9, further comprising, after lacerating the target anatomical structure, deactivating the first radiofrequency electrode and the second radiofrequency electrode.

14. The method of claim 13, further comprising, after deactivating the first radiofrequency electrode and the second radiofrequency electrode, withdrawing the laceration device from a patient's body while the clamp remains in a closed position.

15. A method for perforating and lacerating a target anatomical structure during a transvenous structural heart procedure, comprising:
advancing a clamp of a laceration device towards the target anatomical structure, the clamp having a first clamp arm with a first radiofrequency electrode and a second clamp arm with a second radiofrequency electrode;
positioning the target anatomical structure between the first clamp arm and the second clamp arm;
clamping onto the target anatomical structure by moving the first clamp arm towards the second clamp arm to secure the clamp to the target anatomical structure and to position a first perforation surface of the first radiofrequency electrode facing a second perforation surface of the second radiofrequency electrode with the target anatomical structure therebetween;
activating the first radiofrequency electrode and the second radiofrequency electrode to perforate the target anatomical structure with the first perforation surface and the second perforation surface; and
applying tensile force to the laceration device while the first radiofrequency electrode and the second radiofrequency electrode are activated,
wherein applying tensile force causes a first laceration surface of the first radiofrequency electrode and a second laceration surface of the second radiofrequency electrode to lacerate the target anatomical structure.

16. The method of claim 15, wherein the target anatomical structure comprises a valve leaflet.

17. The method of claim 15, wherein the target anatomical structure comprises an atrial septum.

18. The method of claim 15, wherein clamping onto the target anatomical structure comprises manipulating a clamp actuator connected to the clamp via a shaft of the laceration device.

19. The method of claim 15, wherein activating the first radiofrequency electrode and the second radiofrequency electrode causes the first perforation surface and the second perforation surface to perforate through the target anatomical structure until the first perforation surface and the second perforation surface contact each other.

20. The method of claim 15, wherein the first laceration surface is transverse to the first perforation surface and extends towards the first clamp arm from the first perforation surface, and wherein the second laceration surface is transverse to the second perforation surface and extends towards the second clamp arm from the second perforation surface.
